(19) United States
(12) Patent Application Publication
(10) Pub. No.: US 2008/0003632 A1
(43) Pub. Date: Jan. 3, 2008

(54) Method for the In Vitro Determination of Cellular Uptake of Exogenous and Endogenous Substances Using Nmr Shift Agents and the Magic Angle Nmr Technique

(75) Inventors: Luisella Calabi, Milano (IT); Lino Paleari, Lissone (IT); Luca Biondi, Milano (IT); Mario Demiranda, Cesano Maderno (IT); Goffredo Alfieri, Pontida (IT); Stefano Ghelli, Rubiera (IT)

Correspondence Address: BRACCO RESEARCH USA INC., 305- COLLEGE ROAD EAST, PRINCETON, NJ, 08540, US

(73) Assignee: Bracco Imaging SpA, Milan (IT)

(21) Appl. No.: 10/591,965

(22) PCT Filed: Mar. 22, 2005
(86) PCT No.: PCT/EP05/03058

§ 371 Date: Sep. 7, 2006

(30) Foreign Application Priority Data
Mar. 26, 2004 (EP) 04007388.4

(51) Int. Cl. G01N 33/483

(52) U.S. Cl. 435/29

(57) ABSTRACT

The present invention relates to a method for the in vitro quantitative determination of cellular uptake of exogenous or endogenous substances which method comprises applying MAS-NMR spectroscopy technique to an in vitro cellular sample, in combination with a shift agent. The said method is particularly advantageous as it find general applicability for a variety of substances and cell samples.

### FIELD OF THE INVENTION

The present invention relates to a method for the in vitro quantitative determination of the cellular uptake of exogenous or endogenous substances by means of magnetic resonance techniques.

Said method is particularly advantageous as it can be applied, substantially, to all types of samples including, for instance, human or animal cells, cells culture(s), tissue and organ cells, vegetal cells (including wood and fruits), part of trunks, leaves and food cells of both animal or vegetal origin.

### ABBREVIATIONS USED IN THE DESCRIPTION

For sake of clarity and conciseness, a list of the abbreviations/acronyms most frequently used within the present description is herewith enclosed.

ASA Aetilsalycilic acid

BMS Bulk Magnetic Susceptibility

CA/s Contrast Agent/Agents

CC/s Cellular Compartment/Compartments

CC≠SA Cellular compartment in which the SA is not present

CCSA Cellular compartment in which the SA is present

CP-MAS Cross Polarization Magic-Angle-Spinning

CSA Chemical Shift Anisotropy

DDI Dipole-Dipole Interaction

DSS 2,2-dimethyl-2-silapentane-5-sulfonate

ENDO/s Endogenous naturally occurring substance/substances

ESR Electron Spin Resonance

EXO/s Exogenous substance/substances

EXO_{O} EXO substance present in extra-cellular compartment

EXOᵢ EXO substance present in intra-cellular compartment

HRBC Human Red Blood Cells

HR-MAS High Resolution Magic-Angle-Spinning

LIS Lanthanide Induced Shift

LIS^{ENDO} Lanthanide Induced Shift of Endogenous substance (signal/signals)

LIS^{EXO} Lanthanide Induced Shift of Exogenous substance (signal/signals)

LIS_{O} Lanthanide Induced Shift for substance in the extra-cellular compartment

LISᵢ Lanthanide Induced Shift for substance in the intra-cellular compartment

MAS-NMR Magic-Angle-Spinning Nuclear Magnetic Resonance

MECM Multi-photon Excitation Confocal Microscopy

MR Magnetic Resonance

NCT Neutron Capture Therapy

PDT Photodynamic Therapy

SA/s Shift Agent/Agents

SEM Scanning Electronic Microscope

ρ^{ENDO} [SA]/[ENDO], i.e. it represents the ratio: SA concentration to ENDO concentration

ρ^{EXO} [SA]/[EXO], i.e. it represents the ratio: SA concentration to EXO concentration

[ ] molar concentration

[a] molar concentration of substance a

Bibliographic references included in the description by means of numbers in brackets are also reported in the paragraph entitled “References”.

### BACKGROUND OF THE INVENTION

It is well-known in the art that the quantitative determination of cellular uptake may provide valuable data and information concerning, for instance, bio-availability, effectiveness, resistance and toxicity, of a variety of exogenous or endogenous substances. In the field of cosmetics, for instance, the possibility of determining the cellular uptake by derma cells may provide important data for the development of new cosmetic products possessing high performance and low collateral effects.

In botanics, likewise, the determination of the cellular uptake of EXOs can be useful to assess the exposure of vegetables to dangerous chemical products and, also, to get information about their preservation and care (see, as an example, the determination of the cellular uptake of EXOs by the trees of the big forests).

When considering the medical field, in addition, this kind of determination is of utmost importance as it may provide important information for pharmaceutically active ingredients per se, for instance including effectiveness, bio-availability or toxicity thereof. In this respect, cellular uptake represents one of the milestones on which the whole drug development process is based, starting from the identification of a lead compound, up to the final formulation of the drug ready for administration to human and/or animal beings. Moreover, as cellular uptake may also concern compounds which are void of any pharmacological property per se but, once administered, specifically accumulate into human or animal cells, its measurement is particularly important also in the field of diagnostics wherein cellular uptake is closely related to tissue and/or organ specificity of the CAs being used.

According to this latter aspect, as tissue/organ specificity is usually connected with both effectiveness and toxicity of these CAs, the capability of obtaining reliable cellular-uptake measurements may represent the starting point for the development of new contrast agents able, for instance, to provide high contrast imaging at lower dosages.

Substantial analogous considerations apply for radio-sensitizers to be used in PDT and NCT.

Despite the fact that cellular uptake and organ uptake can be somehow confused, they relate to two different kinds of uptake.

In this respect, cellular uptake provides for the accumulation of a given substance in the intra-cellular compartment. As such, its determination effectively provides a mean to quantify the amount of said substance being entered into the cell.

On the other side, as organ uptake provides for the accumulation of a given substance in the organ as a whole, it does not correspond, or necessarily correspond, to the mean cellular uptake. This is because a given substance may accumulate into the organ, for instance in its extra-cellular compartments, as a consequence of possible binding effects or interactions with the molecules being present on the external layer of the cellular membrane.

From all of the above, there is the need of a robust and reliable method for the in vitro measurement or determination of the cellular uptake of exogenous or endogenous substances in a variety of samples.

In this respect, it should be clear to the skilled person that the said method should find general applicability and preferably rely on the quantitative determination of a given parameter directly linked to the cellular uptake per se, so as to provide a direct measurement of it.

In addition, it should not require sample manipulation so as to avoid as much as possible changes in the concentration of the species at equilibrium or modifications of the functionality of the cellular membrane and of its integrity.

According to our knowledge, the methods currently known in the art do not fulfill all of these requirements.

The known methods being used in the determination of the cellular uptake of exogenous or endogenous substances may be conveniently grouped into two main categories: those requiring sample treatment (group A) and those not requiring sample treatment (Group B).

Group A In principle, the methods belonging to this group can be used to measure the cellular uptake of all types of EXOs and ENDOs in any type of cell or tissue sample. The said methods usually enable to determine the total content of a given substance without differentiating on how this same substance is partitioned in the various CCs.

These methods are based, essentially, on chemical and chemico-physical analytical techniques requiring a sample pre-treatment, aimed to separate the intra-cellular fluid from the rest of the sample, which may vary according to the technique being used and to the type of sample being tested.

Anyway, whichever the treatment is, it generally produces modifications of the cellular system with consequent changes in the concentration of the species present in the cellular compartments at equilibrium, and/or changes of the mechanisms governing the transport of substances across the cellular membrane. Despite any possible misleading result, these effects may thus contribute to render the sample under analysis no more representative of the reality.

When cells are cultured in liquid phase, for instance, the pre-treatment may consist in the separation of the cells from the extra-cellular fluid by means of several washing steps and subsequent centrifugation. This treatment can dramatically change the concentrations at equilibrium and break cells, or a relevant portion of them, with consequent perfusion of the intra-cellular fluid, or part of it, into the extra-cellular compartment.

On the other hand, when cells are cultured on semisolid or solid matrix, an even worst situation can occur because of the vigorous treatment needed to free the cells from the matrix itself. Under these conditions, changes of the concentrations of the species at equilibrium and modifications of cellular membrane functionality and integrity, during sample preparation, are almost inevitable.

The above is even more evident in the case of cell agglomerates or strips of tissues wherein treatments may be particularly drastic and invasive as they are directed to obtain isolated cells through tissutal matrix destruction.

Because of the above drawbacks, the determination of the cellular uptake according to the methods of group A do not appear to provide a reliable representation of the cellular uptake occurred in the original, untreated sample.

Group B Despite the fat that these methods have the common advantage of not requiring the above sample treatments, they are applicable to a few specific substances only. In fact, if the EXO (or ENDO) under determination is a substance containing atoms different from those naturally occurring inside the cells, that is atoms other than hydrogen, carbon, nitrogen, sodium and the like, its concentration inside the cell may be determined by use of a Scanning Electronic Microscope combined with micro-analysis, according to known techniques. However, as most of EXOs and ENDOs are organic molecules, their cellular uptake cannot be measured in this way.

On the contrary, the cellular uptake of heavy metals including, for example, paramagnetic metals, free metal ions and metal complexes thereof, may be all determined by using this technique.

If the EXO is a paramagnetic metal complex, some known methods based on ESR spectroscopy (1) or MAS-NMR spectroscopy (2,3) may be used. When the paramagnetic metal is gadolinium, in particular, the cellular uptake measurement can be also derived from the enhancement of the contrast in MR imaging (4).

The above MAS-NMR technique has been applied to the determination of the permeability of human blood cells by Magnetic Resonance Imaging Contrast Agents (MRI-CAs), particularly polyamino polycarboxylic Gd based contrast agents. This method comprises the use of a lanthanide complex able to produce a clearly detectable lanthanide induced shift (LIS) and a very weak relaxation (line broadening) on NMR signals of intra- and extra-cellular water protons. The rationale for this method relies on the complete isostructurality between the Gd-contrast agents (CA), which intra- or extra-cellular concentration has to be determined, and the lanthanide complex acting as shift agents (LIS agent). In other words, as both CA and LIS agent are supposed to show a very similar behaviour (because of their isostructurality), the actual determinations of where the LIS agent is, i.e. its exact intra- or extra-cellular concentration, are deemed to substantially correspond to where the CA would be and to the CA intra- or extra-cellular concentration, respectively.

As formerly indicated, however, the above method only provides for the determination of the cellular uptake of paramagnetic complexes and, hence, it cannot be applied to the determination of any different substance.

If the EXO is a manganese compound, the cellular uptake measurement can be carried out through the observation of the line broadening of phosphorus signal in ATP ³¹P-NMR spectra (5).

If the EXO under examination provides for a fluorescent spectrum well distinguishable from those produced by naturally occurring substances, i.e. endogenous substances inside the sample, MECM technique (6,7) can be used. However, as ENDOs usually contain several organic chromophores, this technique may only find application in a limited number of situations.

In the case of some endogenous organic metabolites (11), water (12) and free metal ion substances, for instance Na+, K+, Li+and the like, a method based on SAs and NMR techniques (8,9,10) has been used to measure the concentration ratio between intra- and extra-cellular content. Nevertheless, despite the fact that the said concentration ratio is known to be related to the cellular uptake, the above method cannot quantify the single compartmental concentration of these substances.

Moreover, as it does not imply the use of MAS technique, it is not able to address the problems related to the presence of Chemical shift anisotropy, Dipole-Dipole Interactions and BMS as well as problems deriving from any possible overlapping between nuclei signals normally occurring into biological samples and from an incomplete differentiation of the signal with respect to the spectrum base line, i.e. an incomplete “NMR visibility” of the signal.

From all of the above, it appears that the reliability of the obtained measures according to the methods of Group B is insufficient in most of the cases. Importantly, no standardized methodologies can be considered for the methods of group B as too many variables apply including, for instance, the nature of the sample, its handling and the substance under investigation.

Moreover, known in vitro methods of both groups A and B appear to be time consuming and thus imply high costs, mainly because of the huge amount of work needed for the tuning of the method and/or for sample preparation.

Alternative approaches for instance comprising the in vivo determination of cellular uptake have been also disclosed. In this respect, although in vivo data are currently considered the “gold standard”, their reliability is not yet doubt-free as given experiments have shown relevant drawbacks due to long experimental times, high costs mainly due to animals stabling and handling and, also, ethical issues.

Therefore, there is still the need for a reliable and fast method of general applicability enabling the determination of the cellular-uptake for a wide number of substances, in a variety of samples.

### SUMMARY OF THE INVENTION

We have now found a method for assessing the cellular uptake of exogenous or endogenous substances that, advantageously, does not present any of the aforementioned drawbacks.

Therefore, it is a first object of the present invention a method for the in vitro determination of cellular uptake of exogenous or endogenous substances in a cell sample, which method comprises:

1) selecting a suitable shift agent (SA) and nucleus combination for the measurement of cellular uptake of the exogenous or endogenous substance under investigation, through MAS-NMR spectroscopy;

2) determining the cellular compartment/s (CC/s) in which said exogenous or endogenous substance distributes, through MAS-NMR spectroscopy; and

3) measuring the compartmental concentration of the said exogenous or endogenous substance.

In the present description, unless otherwise provided, with the term cellular uptake of a given substance we intend the quantization of the amount of substance entered into the cell, that is to say penetrated into the cell across the cell membrane, independently from its stay in one or more of the cellular compartments.

Unless otherwise indicated, the term “cellular compartment” is herewith intended to include every portion of the cell being delimitated by a membrane.

In the present description, unless otherwise provided, with the term exogenous substance we intend every substance not naturally occurring in a biological sample, that is to say not resulting from a natural biological process, also including pathological processes.

Non limiting examples of exogenous substances according to the invention may thus include exogenous organic substances and exogenous metals or metal ions which NMR signals can be observed.

Preferred exogenous substances according to the invention include, for instance, drugs for human and veterinary use, diagnostic and therapeutics agents, contrast agents for imaging techniques, radio-sensitizers for photodynamic and neutron capture therapy, pesticides including herbicides, fertilizers, food additives, preservatives, cosmetics, colorants, waste products, pollutants, and chemicals in general.

Even more preferred exogenous substances are drugs and therapeutic agents, contrast agents for imaging techniques, radio-sensitizers for photodynamic and neutron capture therapy, pesticides, fertilizers, food additives, colorants, waste products, pollutants, and cosmetics.

Unless otherwise indicated, in the present description the term endogenous substance includes every substance resulting from normal or pathological biochemical processes of cells and tissues. Non-limiting examples of endogenous substances according to the invention may thus include any compound from natural metabolic pathways such as, for instance, natural carbohydrates, urea, lactate, citrate, acetate, carbonate, malonate, choline, creatine, phosphate, piruvate and natural amino acids.

According to a preferred embodiment, the present invention relates to a method for the in vitro determination of cellular uptake of exogenous substances (EXOs).

The method of the invention enables the “non-invasive” measurement of the cellular uptake of a variety of substances in a wide range of in vitro samples, either in liquid or semisolid media, including strips of tissues or organs and even organs as a whole.

In the present description, unless otherwise provided, with the term “non-invasive” we rely to the fact that the in vitro sample is not treated or pre-treated or, alternatively, that any needed treatment or manipulation is particularly light and, hence, does not produce modifications of the concentration of the chemical species at equilibrium or modifications of the processes governing the cellular uptake.

As a result, the method of the invention presents the remarkable advantage that the sample maintains as intact all of its biological functionalities.

In addition, the measurement of a parameter directly linked to the absolute concentration of the analyzed exogenous or endogenous substance in the different CCs guarantees high reliability and reproducibility of the obtained data, thus replacing the need for a large number of in vivo tests.

Moreover, the sensitivity of the measurements is the one typical for NMR spectroscopy: micromolar concentrations are required when medium strength magnetic fields are used and even sub-micromolar concentrations may suffice when high magnetic fields and cryogenic technology for probeheads are used. And also, NMR spectra can be acquired in few minutes and, in addition, the experimental conditions being first tuned for the cellular uptake of a given substance may be conveniently adopted for determining the cellular uptake of other substances. This is because the experimental conditions are mainly dependent from the type of sample under consideration and, to a lesser extent, from the type of substance being tested.

Accordingly, the method of the invention allows easy and fast measurements as well as a high level of standardization because, apart from not requiring complex sample treatment, it is based on a single technique that comprises MAS-NMR spectroscopy in combination with lanthanide SA.

For a better understanding of the invention, the following technical details are now given. For ease of reference, they are specifically addressed to the exogenous substances (EXOs) only but they are applicable as well to the endogenous substances (ENDOs).

As formerly indicated, the method of the invention comprises applying the so-called MAS-NMR spectroscopy in combination with Shift agents (SAs).

With the term MAS-NMR spectroscopy we mean the totality of pulse sequences which can be utilized to acquire NMR spectra with probehead designed for NMR measures with sample in fast spinning at the so-called “Magic Angle”.

The combined application of MAS-NMR spectroscopy, in particular, is the “tool” that consents an enlarged and, at the same time, efficacious use of the SAs as per the method of the invention to measure the cellular uptake of a wide range of substances, in a number of different in vitro samples, so making the method of the invention of general applicability.

The use of a SA for cellular uptake measurements, in fact, is based on and may only be advantageously applied when the signals of interest, corresponding to the given EXO (or ENDO) in the intra- and extra-CCs, are both detectable and well separated, to allow a reliable measure of their areas wherein this means that no overlapping can exist. Moreover, the said signals must be due to the 100% of the EXO (or ENDO) in the sample. This means that the whole signal has to be completely detectable, “visible”, with respect the spectrum base line.

The combined use of MAS technique according to the method of the invention allows an almost total cancellation of CSA and DDI effects that are generally responsible of the strong line width broadening. Accordingly, it allows the registration of NMR spectra having very sharp line widths, that, as above said, is the condition for a successful enlarged use of a SAs, i.e., of the general applicability of the method of the invention. Moreover, the use of MAS technique as per the present invention consents an almost complete reduction of the undetectable signal amount, that is to say of the “not visible” NMR signal, so providing for an improved reliability of obtained results.

Importantly, according to the method of the invention the LIS effect of the EXO NMR signal is the consequence of the sole and direct interaction between SA and EXO, that is to say LIS effect only arises from a dipole-dipole interactions between SA and EXO and its magnitude decays with the square of the distance between SA and the substance interacting with it.

In other words, in the method of the invention the presence of the SA may only determine a shift of the NMR signal of a substance when this same substance is very close to the SA, i.e. when both EXO and SA stay in the same cellular compartment. In this case, the measured LIS is a quantity directly linked to the absolute concentration of the analyzed EXO in the different CCs, and its value is proportional to the ratio between the EXO and SA concentrations, hereinafter indicated as ρ^{EXO}=[SA]/[EXO].

However, as the above equation, as said, correctly applies only when the observed LIS effect is only due to a direct dipole-dipole interactions occurring between SA and EXO staying in the same cellular compartment, it is necessary that any anisotropic component of Bulk Magnetic Susceptibility (BMS) must be totally cancelled.

As reported in the literature (13-16), in fact, BMS could give rise to LIS across the cellular membrane separating the different cellular compartments, i.e. a LIS effect would also exist between SA and EXO substances not staying in the same cellular compartment. The use of MAS technique according to the method of the invention allows for a complete elimination of any anisotropic component of Bulk Magnetic Susceptibility shift and thus provides for an induced shift only when due to a direct interaction occurring between SA and EXO staying in the same CC.

Moreover, as above said, by means of MAS technique the NMR lineshape in biological samples results much sharper than that in spectra obtained without MAS, thus allowing for a better differentiation between signals and optimal detection of LIS induced by SA on the EXO signals.

From all of the above, after the addition of SA to the sample, the EXO NMR signals may remain unchanged or, alternatively, may undergo to LIS. Then, in the case of EXO unshifted signals, EXO itself stays in CCs not shared or anyway occupied by the SA; on the contrary, in the case of EXO shifted signals, EXO occupies the same CCs of SA. Based on that, a direct measurement of the cellular uptake of the EXO may be thus obtained.

Interestingly, whether unshifted and shifted EXO NMR signals exist in the same sample, the ratio between the areas of both signals directly provides for the distribution ratio of the EXO between the CCs, thus allowing a direct measurement of the EXO cellular uptake.

According to the method of the invention, step (1) above is carried out by:

a) identifying a set of possible SA candidates for said SA and nucleus combination, on the basis of the LIS produced on at least one NMR signal belonging to said EXO;

b) identifying a set of possible candidates for said SA, on the basis of the CC/s in which they distribute; and

c) selecting said SA and nucleus combination, on the basis of the information gathered from steps (a) and (b).

Step (a): Selection of the Shift Agent

In principle, all of the substances containing a paramagnetic nucleus may suitably act as SA according to the method of the invention. Particularly preferred, however, are SA including a lanthanide metal ion.

Even more preferred are lanthanide complexes wherein the ligand is selected from the group consisting of: EDTA (ethylenediaminetetraacetic acid); PCTA (3,6,9,15-tetraazabicyclo-[9.3.1]-pentadeca-1(15)11,13-triene-3,6,9-tris (methane phosphonic) acid); BOPTA ((4RS)-[4-carboxy-5,8,11-tris (carboxymethyl)-1-phenyl-2-oxa-5,8,11-triazatridecan-13-oic acid]) or derivatives thereof; DTPA (diethylenetriamine pentaacetic acid) or derivatives thereof; DOTA (1,4,7,10-tetraazocyclo-dodecane-N,N′,N″,N″″-tetraacetic acid) or derivatives thereof; DO3A (1,4,7,10-tetra azacyclododecane-1,4,7-triacetic acid) or derivatives thereof; DOTP (1,4,7,10-tetraazacyclododecane-1,4,7,10-tetrakis (methane phosphonic) acid or derivatives thereof; ([3β(R),5β,12α:]-3-[[4-[bis[2-bis(carboxymethyl)amino]-ethyl]amino]-4-carboxy-1-oxobutyl]amino]-12-hydroxycholan-24-oic acid). Preferred metal ions of the lanthanide group include: Ce³⁺; Pr³⁺; Nd³⁺; Pm³⁺; Sm³⁺; Eu³⁺; Tb³⁺; Dy³⁺; Ho³⁺; Er³⁺; Tm³⁺; Yb³⁺.

Step (a): Selection of Nucleus Combination

When referring to nucleus combination and its selection we intend the most suitable nucleus, among those of the EXO substance, being capable of providing an easily detectable EXO NMR signal and an equally easily detectable induced shift on that signal, by the action of the SA. Accordingly, preferred nuclei as per the method of the invention, are those allowing an easy detection of the EXO NMR signal by use of MAS-NMR technique.

Usually, proton and phosphorus do not represent the better choice as ¹H-NMR and ³¹P-NMR spectra of biological samples frequently show strong overcrowding and peaks overlapping, due to various interfering substances naturally occurring in the sample.

As an example, when exogenous substances under investigation are available in a suitable isotopically enriched form, preferred nuclei may thus include ¹³C and ¹⁵N.

More generally, however, preferred nuclei may include those which are not present in the natural biological samples such as fluorine-19, deuterium, and boron-11.

To identify the most suitable set of SAs and nuclei combination for the quantitative determination of the cellular uptake of a given EXO, the said EXO is dissolved in D₂O and, by employing a variety of combinations of different SAs with different nuclei and by varying the ratio ρ^{EXO}=[SA]/[EXO], LIS^{EXO} signals are thus measured. SAs and nuclei combinations inducing the largest LIS^{EXO} signals are those most suitable for use with the EXO under investigation. Said largest LIS^{EXO} signal/s, hereinafter referred to as marker^{EXO} signal/s, has/have to be considered as preferred according to the invention.

Step (b): Determination of [SA]_{CC}.

To carry out the method of the invention it is necessary to know with a high degree of precision, at priori or by experimental measures, the concentration of SA into the different Cellular Compartments (i.e., [SA]_{CC}).

If experimental measurements are required, any one of the methods known in the art and concerning paramagnetic complexes can be used (see, as a reference, any of the aforementioned methods listed in Group B).

When using the method based on MAS-NMR spectroscopy (2,3), in particular, all of the steps 1-3 of the method of the invention may be experimentally carried out by using this same technique.

Step (c): Selection of the SA and Nucleus Combination.

In principle, suitable SA for measuring EXO cellular uptake should be able to induce the largest LIS^{EXO} signal of at least one of the EXO NMR signals (the marker^{EXO} signal). Although not mandatory, SAs distributing in one Cellular Compartment only have to be considered as preferred because allowing easy calculations. By combining the results obtained from previous steps (a) and (b) it is thus possible to select the optimal SA and nucleus combination. Frequently, however, the chosen combination may just represent the best compromise among the different indications resulting from steps (a) and (b).

According to the method of the invention, step (2) above is carried out by:

d) acquiring the MAS-NMR spectrum of the in vitro sample containing the EXO under investigation and determining the marker^{EXO} signal/s;

e) adding a suitable amount of the selected SA to the above in vitro sample, so as to induce a significant LIS of marker^{EXO} signal/s, and re-acquiring the same MAS-NMR spectrum; and

f) comparing the marker^{EXO} signal/s of steps (d) and (e) and determining in which Cellular Compartment the EXO is present.

The MAS-NMR spectra can show different situations, as per the enclosed FIGS. 1-3 (a detailed explanation of all of the enclosed figures is also reported before the experimental section).

The use of a preferred SA staying in one of the CCs only is represented in FIGS. 1 and 2.

FIG. 1—traces a-f.

Trace a shows a marker^{EXO} signal (in this case it is a single, one component signal) recorded in the absence of SA.

Following the addition of SA:
the marker^{EXO} signal remains unchanged (trace b). In this case all of EXO stays in a CC different from that in which SA stays, i.e. all of the EXO is EXO_{CC≠SA;}
the marker^{EXO} signal remains as a single peak but shifts with respect to the original position (traces c or d). In this case the EXO and SA stay in the same CC, i.e. all of the EXO is EXO_{CCSA};
the marker^{EXO} signal splits in at least two components (traces e or f). The EXO corresponding to the shifted signal is EXO_{CCSA} whilst the EXO corresponding to the unshifted signal is EXO_{CC≠SA}. The ratio between the areas of the two signals is proportional to the ratio between [EXO_{CCSA}] and [EXO_{CC≠SA}].

It is worth noting that situations corresponding to trace e or f may appear as per the situation of trace b. This may happen when the LIS of marker^{EXO} signal is too small, i.e. insufficient to separate the EXO_{CCSA} signal from the one due to the EXO_{CC≠SA}.

Therefore, in the case of spectra like the one of trace b, it may be convenient to add an additional amount of SA to the sample, so as to check the optional existence of two or more overlapping signals.

FIG. 2—traces a-f.

Trace a shows a marker^{EXO} signal having two (or many) components, in the absence of SA. These components may be due to the presence of the EXO in different CCs or to the fact that the EXO has two marker^{EXO} signals, for example as a consequence of two different chemical specie such as isomers or conformers, or because of the possible interaction with endogenous substances, membrane and the like.

Following the addition of SA:
each marker^{EXO} signal remains unchanged (trace b). In this case the EXO stays in CCs different from that where SA stays, i.e. all of the EXO is EXO_{CC≠SA};
some marker_{EXO} signals remain unchanged whilst other shift: signal A shifts while signal B remain unchanged (trace c and d) or vice-versa (traces e and f). In this case the shifted signals correspond to EXO_{CCSA} whilst the other correspond to EXO_{CC≠SA};
all of the marker^{EXO} signals shift. In this case all of the EXO is EXO_{CCSA}.

It is worth noting that situations corresponding to trace c-f may appear as per the situation of trace b. This may happen when the LIS of marker^{EXO} signal is too small, i.e. insufficient to separate the EXO_{CCSA} signal from the one due to the EXO_{CC≠SA}.

Therefore, in the case of spectra like the one of trace b, it may be convenient to add an additional amount of SA to the sample, so as to check the optional existence of two or more overlapping signals.

From all of the above, if in step (b) it has been established that SA only stays in the extra-cellular compartment (o), it is possible to affirm that EXO_{CCSA}=EXO_{O} and EXO_{CC≠SA}=EXOᵢ. Vice-versa, if in step (b) it has been found that SA only stays in the intra-cellular compartment (i), it is possible to affirm that EXO_{CCSA}=EXOᵢ and EXO_{CC≠SA}=EXO_{O}.

The use of a preferred SA being shared among many compartments is reported in FIGS. 3 and 4.

FIG. 3—traces a-h.

Trace a shows a marker^{EXO} signal, showing only one component, in the absence of SA.

Following the addition of SA:
the marker^{EXO} signal remains unchanged (trace b). In this case the EXO stays in one or more CC/s anyway different from the one where SA stays. The EXO is all EXO_{CC≠SA};
the marker^{EXO} signal shifts (traces c-d). In this case the EXO is all EXO_{CCSA}; and SA stays in one CC only (or in different CCs in case marker^{EXO} are isochronous);
the marker^{EXO} signal shifts and the peak marked with an asterisk (*) remains unshifted (traces e-f). In this case the shifted signals correspond to EXO_{CCSA} and signal (*) correspond to EXO_{CC≠SA};
the marker^{EXO} signal splits into two or more components and peak (*) remains unshifted (traces g-h). In this case the shifted signals correspond to EXO_{CCSA} and signal (*) corresponds to EXO_{CC≠SA}.

FIG. 4—traces a-h.

Trace a shows a marker^{EXO} signal showing two (in general many) components in the absence of SA.

Following the addition of SA:
the marker^{EXO} signals remain unchanged (trace b). In this case the EXO stays in one or more CC different from that where SA stays. The EXO is all EXO_{CC≠SA};
one signal remains unchanged whilst the other shifts (trace c, d, e, f). It may also occur that the shifted signal splits into two or more components. The shifted signals correspond to EXO_{CCSA} and the unshifted signal corresponds to EXO_{CC≠SA};
all of the signals shift (trace g, h). In this case all of the EXO is EXO_{CCSA}.

Since the CCs where SA distributes are known, upon comparison between the marker^{EXO} signal/s in the absence and in the presence of SA, the CC/s where EXO stays may be also determined.

The determination of the EXO compartmental concentration as per step (3) of the method of the invention is carried out according to the following possible situations.
if all of the EXO is EXO_{CC≠SA} (as per FIGS. 1b; 2b; 3b; 4b), [EXO_{CC≠SA}] is obtained by considering the amount of EXO being added to the sample and the volume of the CC where EXO stays [CC is known from former step (b)].

The values of [EXO_{CC≠SA}] may also be determined by use of the equation ρ^{EXO}=[SA]/[EXO], because [SA]_{CC} can be known. The value of ρ^{EXO} may be determined trough the graph of LIS^{EXO} vs. ρ^{EXO}. From [EXO_{CC≠SA}], by knowing the volume of the CC, the amount of EXO_{CC≠SA} may be obtained.

The said graph can be obtained by acquiring a series of MAS-NMR spectra of the known EXO under investigation in a medium as similar as possible to the one of the in vitro sample such as, for instance, a physiological solution or plasma, and by using different known [SA]. At each variation of [SA]/[EXO], LIS^{EXO} signal is then measured.
if all of the EXO is EXO_{CCSA} and SA stays in one CC only (as per FIGS. 1c; 1d; 2c; 2d), the two methods just disclosed for the determination of [EXO_{CC≠SA}] may also be suitably applied for the determination of [EXO_{CCSA}] and the amount of EXO_{CCSA}.
if EXO is partitioned as EXO_{CCSA} and EXO_{CC≠SA}, SA stays in one CC only (as per FIGS. 1e; 1f; 2e; 2f) and the amount of EXO added to the sample is known, the ratio between the area of the peaks, corresponding to EXO_{CCSA} and EXO_{CC≠SA}, just supplies the ratio [EXO_{CCSA}]/[EXO_{CC≠SA}]. In this case, if the volume of at least one of the CCs is know, the amounts of [EXO_{CCSA}] and [EXO_{CC≠SA}] result thus determined. On the contrary, if CCs volumes are not know, [EXO^{CCSA}] can be determined as described in the previous case, by using LIS^{EXO} signal vs. ρ^{EXO} and, consequently, also [EXO_{CCSA}] results to be determined.
if EXO and SA distribute into more than one CC (as per FIGS. 3c-3h; 4c-4h), the possibility of determining all of the EXO compartmental concentrations may require additional stoichiometric calculations which complexity may vary for the different situations, depending from the availability of some or all of the CC volumes and the number of CCs where SA and EXO distribute. In any case, by means of LIS^{EXO} signal vs. ρ^{EXO} graph, the values of the EXO compartmental concentrations can be calculated. To sum up, step (3) of the method of the invention is carried out by taking into account the CC/s where EXO stay, the volume/s of said CC/s, the value/s of the area/s under the marker^{EXO} signal/s, the calculated ρ^{EXO} for every CC in which EXO stays, and by solving the system of equations connecting these parameters.

This situation is disclosed, in more details, in the subsequent experimental section (see example 1).

The method of the invention may be advantageously used in a variety of fields such as, for instance:
in medicine, for the screening of bio-availability, effectiveness, resistance and toxicity of exogenous substances including, for example, drugs for human and veterinary use, diagnostic contrast agents and radio-sensitizer for photodynamic and neutron capture therapy as well as in diagnosis and care of diseases and in drugs therapy follow-up and o for the study of metabolism related to pathologies and thereof care.
in the pharmacological field, for the screening and development of drugs starting, for example, from the identification of a lead compound up to the final formulation of the drug;
in toxicology, to assess the cellular uptake and, hence, the exposure to chemicals, in particular of pesticides, fertilizer, pollutants and waste products;
in the consumer field, to determine the cellular uptake of exogenous substances such as, food additives, artificial colourings and preservatives;
in cosmetics, to determine the cellular uptake of compounds by derma cells and, more in general, for the development of new products endowed with better performance and lower collateral effects;
in diagnostics, to assess the tissue and/or organ specificity of the used CAs wherein said values are usually connected both with effectiveness and toxicity of these substances;
in pharmacokinetics, to assess and study the kinetic parameters governing the cellular uptake;
in botanics, to determine the exposure of vegetables to dangerous chemical products and, also, in the preservation and care of the vegetables themselves.

### EXPLANATION OF THE FIGURES

FIGS. 1 to 4 represent MAS-NMR spectra as per the method of the invention, in a variety of situations. More in particular,

FIG. 1 represents a marker^{EXO} signal showing a single peak in the absence of SA. Trace a: spectrum in the absence of SA; trace b-f spectra in the presence of SA staying in one CC only.

FIG. 2 represents a marker^{EXO} signal showing more than one peak, in the absence of SA. Trace a: spectrum in the absence of SA; trace b-f: spectra in the presence of SA staying in one CC only.

FIG. 3 represents a marker^{EXO} signal showing a single peak, in the absence of SA. Trace a: spectrum in the absence of SA; trace b-h: spectra in the presence of SA staying in more than one cellular compartments.

FIG. 4 represents a marker^{EXO} signal showing more than one peak, in the absence of SA. Trace a: spectrum in the absence of SA; trace b-h: spectra in the presence of SA staying in more than one cellular compartments.

FIG. 5 represents the ¹H NMR spectrum of acetylsalicylic acid in D₂O.

FIG. 6 represents the graph of LIS^{ACETYLSALICYLIC ACID} vs. ρ^{ACETYLSALICYLIC ACID}=[Dy-BOPTA]/[ACETYLSALICYLIC ACID]

FIG. 7 represents the ¹H MAS-NMR spectrum of acetylsalicylic acid in HRBC suspension. Trace a: spectrum of HRBC; trace b: same sample of trace a but after addition of acetylsalicylic acid (100 μl, 1M stock solution); trace c: same sample of trace b but after addition of Dy-BOPTA (80 μl, 0.1M stock solution).

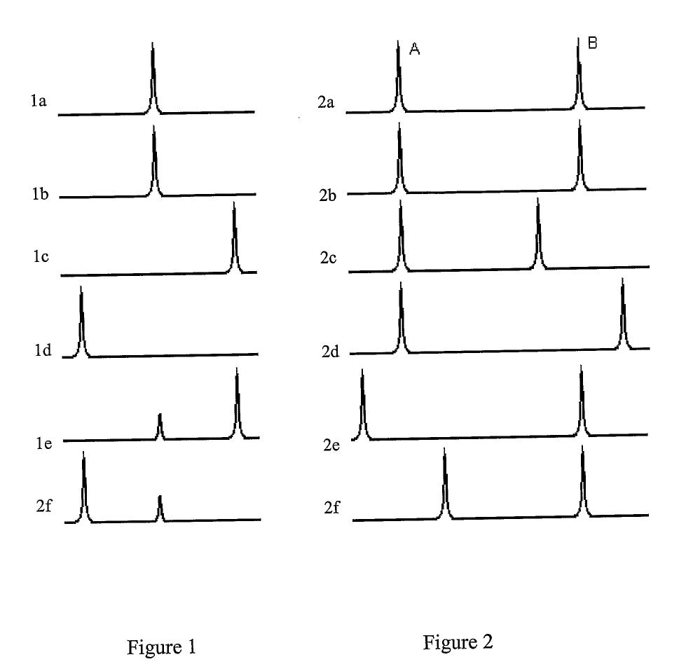

### EXPERIMENTAL SECTION

With the aim of better illustrating the present invention, without posing any limitation to it, the following examples are now given.

### EXAMPLE 1 (Theoretical)

A given SA distributes into three different CCs (hereinafter named as CC1, and CC2) and three shifted marker^{EXO} signals exist. The values of [SA_{CC1}] [SA_{CC2}] are known from previous step (b) of the method of the invention.

From NMR-MAS spectra the values of LIS^{EXO}_{CC1}, and LIS^{EXO}_{CC2} signals can be measured and by the graphs of LIS^{EXO} signal vs. ρ^{EXO}, the corresponding values of ρ^{EXO}_{CC1}, ρ^{EXO}_{CC2}, are obtained. Moreover, the areas A₁ and A₂ of the two marker^{EXO} signals can be measured by integration of the NMR spectrum. Being [SA_{CC}] known, it is possible to determine the [EXO_{CC}] by the following set of equations:

$$\begin{matrix}\begin{matrix}[ {EXO}_{CC 1} ] = [ {SA}_{CC 1} ] / {ρ}_{CC 1}^{EXO} \\ [ {EXO}_{CC 2} ] = [ {SA}_{CC 2} ] / {ρ}_{CC 2}^{EXO}\end{matrix} ) & equation set no . 1 \\ \begin{matrix}[ {EXO}_{CC 1} ] / [ {EXO}_{CC 2} ] = {A}_{CC 1} / {A}_{CC 2} \\ [ {EXO}_{CC 1} ] / [ {EXO}_{CC 3} ] = {A}_{CC 1} / {A}_{CC 3}\end{matrix} ) & equation set no . 2\end{matrix}$$

To solve the system, it is necessary to calculate all of the possible combinations and verify which [EXOs] are congruent with both sets of equations 1 and 2.

To better clarify what above reported, this same example is also expressed through numerical values.

Let us suppose to have the compartmental distribution reported in Table 1 and to label the two different compartments as 1 and 2. The experimental data are:
from the measured LIS: ρ₁=0.02; ρ₂=0.093
from previous step (b): [SA_{CC1}]=0.2; [SA_{CC2}]=1.4;
from MAS-NMR spectra calculation of the areas provides for:
A₁=100; A₂=150.66

From these values it is possible to calculate (by equation set 1) all of the possible values of [SA_{CC}]/ρ^{EXO} i.e. [EXO_{CC}], in the three compartments (see values reported in Table 2).

From these [EXOs] values it is possible to calculate all of the values of the left terms of equation set 2, for all of the possible combinations (see data reported in Table 3). Then, it is possible to calculate the right terms of equation set 2, for all of the possible combinations, by using the experimental values of the signal areas (see data reported in Table 4).

TABLE 1

| | SA | EXO | ρ | signal area |
|---|---|---|---|---|
| [SA_{CC1}] | 0.2 | 10 | 0.02 | 100 |
| [SA_{CC2}] | 1.4 | 15 | 0.093 | 150 |

Total added mM of SA = 1.6, total added mM of EXO = 25.

TABLE 2

| | [SA_{CC1}] | [SA_{CC2}] |
|---|---|---|
| ρ₁ | [EXO_{CC1}] = 10.00 | [EXO_{CC2}] = 70.00 |
| ρ₂ | [EXO_{CC1}] = 2.15 | [EXO_{CC2}] = 15.00 |

TABLE 3

| | | |
|---|---|---|
| [EXO_{CC1}]/[EXO_{CC2}] | 0.14 | 0.67 |
| | 0.03 | 0.14 |

TABLE 4

| | A₁ | A₂ |
|---|---|---|
| A₁ | — | 1.50 |
| A₂ | 0.67 | — |

The value reported in Tables 3 which fit with this in Table 4, are highlighted in bold characters. They correspond to:

[EXO_{C1}]/[EXO_{C2}]=0.67 obtained by [EXO_{C1}]=10 and [EXO_{C2}]=15

A₁/A₂=0.67 obtained by A₁=100 and A₂=150

therefore: [EXO_{C1}]=10; [EXO_{C2}]=15;
A_{C1}=100; A_{C2}=150

Accordingly, the solution of the system has allowed the calculation of the values of [EXO_{CC1}] and [EXO_{CC2}].

### EXAMPLE 2

Determination of Cellular Uptake of Acetylsalicylic Acid in Red Blood Cells

In this example, EXO=Acetylsalicylic acid; SA=Dy-BOPTA (Dy is the symbol of Dysprosium, one of the lanthanides known as shift agent).

The nucleus used to determine Acetylsalicylic acid cellular uptake is the proton.

The in vitro determination has been preformed on HRBC obtained by human blood treated as described below.

Centrifugation: the employed centrifuge was HERAEUS SEPATECH OMNIFUGE 2 ORS, rotor model 3360. Centrifugation was done at 2109 g (equivalent to 3500 rpm) at 4° C. for 15 minutes.

Living HRBC preparation: human blood (to which sodium citrate has been added as an anticoagulant) was centrifuged. After that, HRBC pellets were separated from serum and white cell interface, carefully obtaining a solution of red cells free of white cells; accordingly, red cells with 80% hematocrit were obtained.

Dy-BOPTA stock solution: a 0.1 M stock solution of Dy-BOPTA was used in all of the measurement to obtain the suitable [SA].

Acetylsalicylic acid stock solution: a 1 M stock solution of ASA containing 0.1 M of DSS (as internal standard for quantitative determination), was used in all of the measurements.

The solution was prepared by dissolving 180.16 mg of ASA and 23.63 mg of DSS in 200 ul of H2O, adding NaOH 2N until dissolution, lowering, if necessary, the pH to 7 with HCl 1N, then filling the volume to 1 ml. In this way two forms of ASA are generated that are below indicated as A and B.

Acetylsalicylic acid in HRBC: a sample containing 1 ml of HRBC (80% hematocrit), 100 μmoles of acetylsalicylic acid (i.e. 100 μl of acetylsalicylic acid stock solution) and 8 μmoles of Dy-BOPTA (i.e. 80 μl of Dy-BOPTA stock solution) was employed to measure the uptake of acetylsalicylic acid in HRBC.

Proton ¹H NMR spectra: all the ¹H NMR spectra have been acquired on a Bruker AMX 600 SB spectrometer at the frequency of 600.13 MHz. A multinuclear HR-MAS probehead with double bearing and 4 mm rotor with 12 μl spherical insert have been employed. Experimental conditions were: sample rotating speed=3500 Hz; spectra width=12,000 Hz (c.a. 20 ppm); time domain data points=128 K; number of scans=16; pulse length=11.7 μs; recycle delay=10 s; CPMG sequence; Fourier Transform by 0.5 Hz of enhancement multiplication function, sample temperature=25° C.

Dy-BOPTA employed in the present experiment has been selected from other Dysprosium chelates as preferred SA because it is known from the literature that it does not penetrate into HRBC (2).

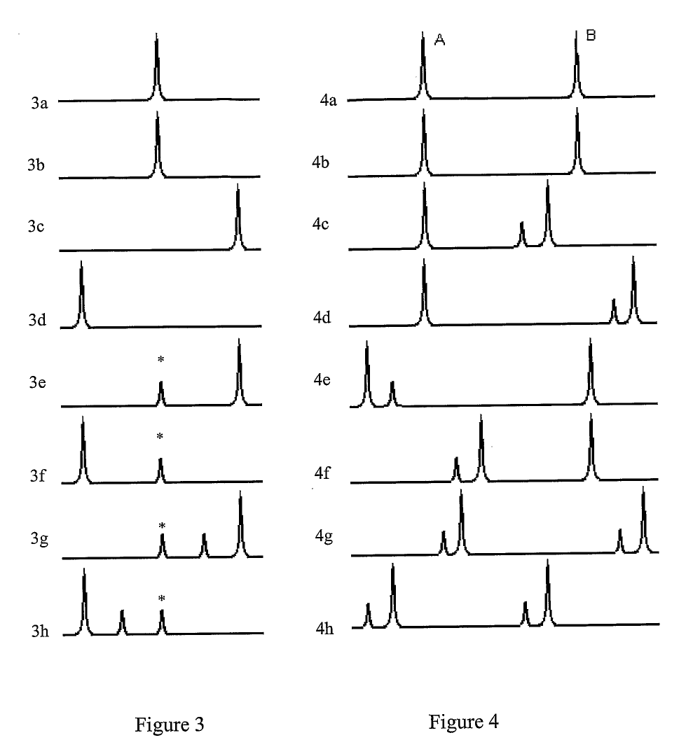

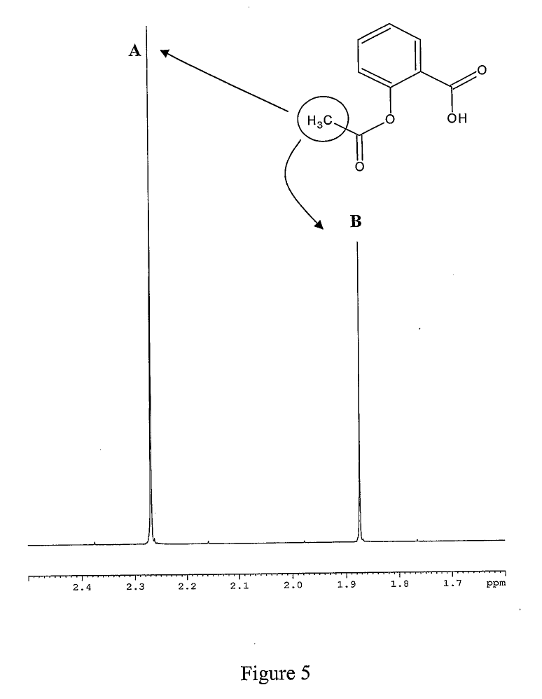

Proton spectra have been employed to determine acetylsalicylic acid cellular uptake, because ¹H is the sole nucleus present in acetylsalicylic acid having a high NMR sensitivity. The ¹H NMR spectra of acetylsalicylic acid in water showed two signals (A and B), in correspondence to the methyl group, as per the enclosed FIG. 5. This case is similar to the one shown in FIG. 2 trace a. Both of the two signals are marker^{EXO} signals.

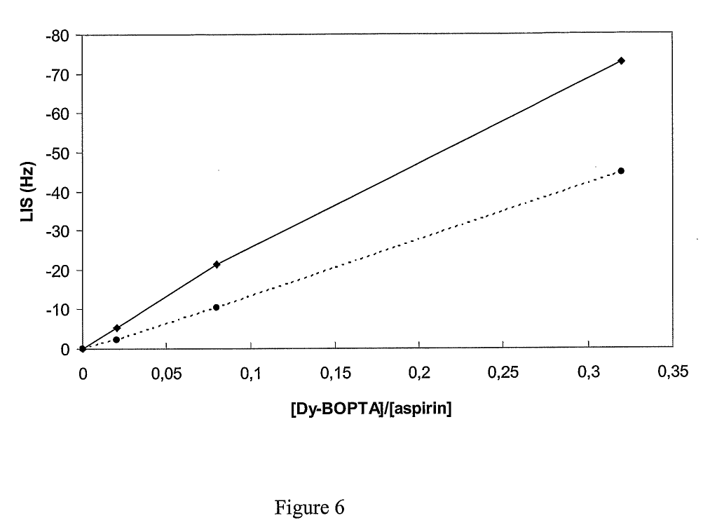

The calculated graph of LIS^{ACETYLSALICYLIC ACID} vs. ρ^{ACETYLSALICYLIC ACID}=[Dy-BOPTA]/[ACETYLSALICYLIC ACID] is reported in FIG. 6, for both the marker^{EXO} signals.

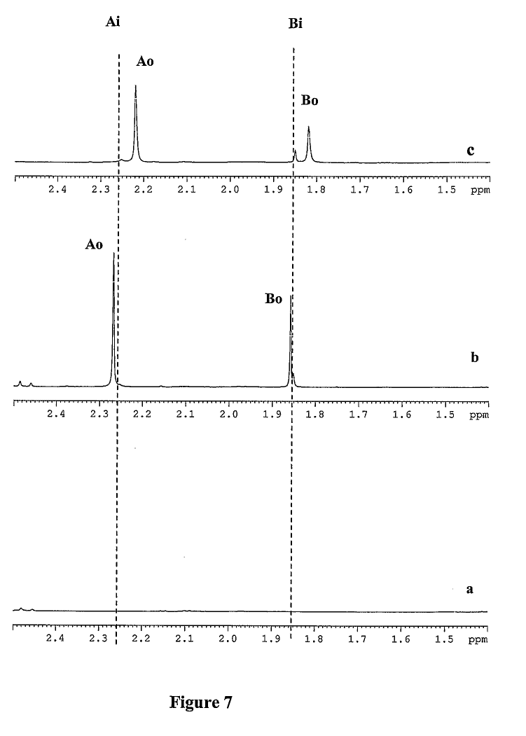

The measurement of cellular uptake of acetylsalicylic acid by HRBC was calculated from the spectra reported in the enclosed FIG. 7.

Firstly, the ¹H NMR-MAS spectrum of HRBC was acquired (FIG. 7—trace a) in the absence of either acetylsalicylic acid (EXO) or of Dy-BOPTA (SA).

Then, 100 μl of acetylsalicylic acid 1M stock solution were added and the spectrum repeated (FIG. 7—trace b). In spectrum of trace b the two components for each signal A and B of the methyl group of acetylsalicylic acid were well visible, i.e. four peaks exist in the proton MAS-NMR spectrum. The splitting of signals A and B induces to suppose that the molecules of acetylsalicylic acid in the extra- and intra-cellular compartments are in magnetically different environments, i.e. the four observed signals corresponded to signals A e B into intra- and extra-cellular compartments, respectively.

Lastly, 80 μl of Dy-BOPTA stock solution were also added to the sample and the spectrum were again re-acquired (FIG. 7—trace c). At this time the four peaks showed a larger difference in their chemical shift with respect to trace b. Two components resulted unshifted with respect to the original marker^{EXO} signals while the other two components were shifted.

Acetylsalicylic acid corresponding to the unshifted marker^{EXO} signals is ACETYLSALICYLIC ACID_{CC≠SA}, i.e. acetylsalicylic acid in the intra-cellular compartment.

On the contrary, acetylsalicylic acid corresponding to the shifted marker^{EXO} signals is ACETYLSALICYLIC ACID_{CCSA}, i.e. acetylsalicylic acid in the extra-cellular compartment.

The ratio between the areas of the two sets of peaks, directly gives the ratio between acetylsalicylic acid in the two cellular compartments for each type A and B. Since the volume of the two cellular compartments can be calculated, it is possible to obtain the absolute amounts of acetylsalicylic acid in the two cellular compartments.

Data:

Peak area of marker^{EXO}(A)ᵢ=5.84

Peak area of marker^{EXO}(A)ₒ=88.28

Peak area of marker^{EXO}(B)ᵢ=11.13

Peak area of marker^{EXO}(B)ₒ=45.50

Volume of intracellular fluid=0.8 ml

(because 1 ml of HRBC with 80% hematocrit were used)

Volume of extracellular fluid=0.2 ml+0.1 ml+0.08 ml=0.38 ml

(0.2 ml=extracellular fluid in HRBC; 0.1 ml=volume of added acetylsalicylic acid solution; 0.08 ml=volume of added SA solution)

Total of added acetylsalicylic acid=100 μmoles

Calculations are the following:

μmoles acetylsalicylic acidᵢ (A)=5.84×100/(5.84+88.28+11.13+45.50)=3.87

μmoles acetylsalicylic acidₒ (A)=88.28×100/(5.84+88.28+11.13+45.50)=58.56

μmoles acetylsalicylic acidᵢ (B)=11.13×100/(5.84+88.28+11.13+45.50)=7.38

μmoles acetylsalicylic acidₒ (B)=45.50×100/(5.84+88.28+11.13+45.50)=30.19

concentration of acetylsalicylic acidᵢ (A)=3.87/0.8=4.83 mM

concentration of acetylsalicylic acidₒ (A)=58.56/0.38=154.10 mM

concentration of acetylsalicylic acidᵢ (B)=7.38/0.8=9.22 mM

concentration of acetylsalicylic acidₒ (B)=30.19/0.38=79.44 mM

[acetylsalicylic acid]ₒ/[acetylsalicylic acid]ᵢ (A)=154.10/4.83=31.90

[acetylsalicylic acid]ᵢ/[acetylsalicylic acid]ᵢ (B)=79.44/9.22=8.61

From all the above, the cellular uptake of the acetylsalicylic acid is:

% of cellular uptake of acetylsalicylic acid A=4.83×100 (4.83+154.10)=3.04%

% of cellular uptake of acetylsalicylic acid B=9.22×100 (9.22+79.44)=10.40%

% of cellular uptake of total acetylsalicylic acid=(4.83+9.22)/(4.83+154.10+9.22+79.44)=5.67%

### BIBLIOGRAPHIC REFERENCES

1. Zaplatin N., Baker K. A., Kleinhans. F. W., Effectiveness and Toxicity of Several DTPA Broadening gents for Biological ESR Spectroscopy, J. Magn. Res. Ser. B 110, 249-254 (1996).
2. Calabi L., Alfieri G., Biondi L., De Miranda M., Paleari L., Ghelli S.—Application of high resolution magic angle spinning NMR spectroscopy to define the cell up-taking of MRI Contrast Agents J. Magn. Reson. 156, 222-229 (2002).
3. Calabi L., Paleari L., Biondi L., Linati L., De Miranda M., Ghelli S., Application of ¹H and ²³Na Magic Angle Spinning NMR Spectroscopy to define the HRBC up-taking of MRI contrast agents, accepted on J. Magn. Reson. 164, 28-34 (2003).
4. Artemov D, Solaiyappan M, Bhujwalla Z M, Magnetic resonance pharmacoangiography to detect and predict chemotherapy delivery to solid tumors, Cancer Res 61:7, 3039-44 (2001).
5. Colet J. M., Vander Elst L, Muller R N, Dynamic evaluation of the hepatic uptake and clearance of manganese-based MRI contrast agents: a 31P NMR study on the isolated and perfused rat liver. J Magn. Reson. Imaging 8:3 663-9 (1998).
6. Chirico G., Cannone F., Olivini F., Beretta S., Baldini G., Diaspro A., Robello M., “A combined confocal and spectroscopic TPE architecture for the identification of single fluorescent molecules”, SPIE proc., Multiphoton Microscopy in the Biomedical Sciences, A. Periasamy, P. T. C. So, Eds., vol. 4262, p. 407-413 (2001).
7. Chirico G., Cannone F., Beretta S., Baldini G., Diaspro A., “Single Molecules Studies By Means Of The Two-Photon Fluorescence Distribution”, Microsc. Res. Tech., (2001) 55:359-364.
8. Degani H., Elgavish G. A., Ionic Permeabilities of Membranes, FEBS Lett. 90(2), 357-360 (1978).
9. Gupta R. K., Gupta P., Direct Observation of Resolved Resonances from Intra- and Extracellular Sodium-23 Ions in NMR Studies of Intact Cells and Tissues Using Dysprosium(III)tripoly phosphate as Paramagnetic Shift Reagent, J. Magn. Reson. 47, 344 - 350 (1982).
10. Miller S. K., Chu W. J., Pohost G. M., Elgavish G. A., Improvement of Spectral Resolution in Shift-Reagent-Aided 23Na NMR Spectroscopy in the Isolated Perfused Rat Heart System, Magn. Reson. Med. 20, 184-195 (1991).
11. Aime S, Botta M, Mainero V, Terreno E., Separation of intra- and extracellular lactate NMR signals using a lanthanide shift reagent. Magn Reson Med 47:1 10-3 (2002).
12. Humpfer E., Spraul M., Nicholson A. W., Nicholson J. K., and Lindon J. C., Direct Observation of Resolved intra- and extracellular Water Signals in Intact Human Red Blood Cells Using 1H MAS NMR Spectroscopy, Magn. Res. Med. 38, 334-336 (1997).
13. Springer C. S. Jr., Physicochemical Principles Influencing Magnetopharmaceuticals in “NMR in Physiology and Biomedicine” (R. J. Gillies, ed.), pp. 75-99, Academic Press Inc., Orlando (1994).
14. Live D. H., and Chan S. I., Bulk Susceptibility Corrections in Nuclear Magnetic Resonance Experiment Using Superconducting Solenois, Anal. Chem. 42(7), 791-792 (1970).
15. Pályka, W. Huang, and C. S. Springer Jr., The Effects of Bulk Magnetic Susceptibility in NMR, Bull. Magn. Reson. 17, 46-53 (1995).
16. Shachar-Hill Y., Berfroy D. E., Pfeffer P. E., and Ratcliffe R. G., Using Bulk Magnetic Susceptibility to Resolve Internal and External Signals in the NMR Spectra of Plant Tissues, J. Magn. Reson. 127, 17-25 (1997).

1. A method for the in vitro determination of cellular uptake of exogenous or endogenous substances in a cell sample, which method comprises:
1) selecting a suitable shift agent (SA) and nucleus combination for the measurement of cellular uptake of the exogenous or endogenous substance under investigation, through MAS-NMR spectroscopy;
2) determining the cellular compartment/s (CC/s) in which said exogenous or endogenous substance distributes, through MAS-NMR spectroscopy; and
3) measuring the compartmental concentration of the said exogenous or endogenous substance.

2. The method according to claim 1 wherein step 1) is carried out by:
a) identifying a set of possible SA candidates for said SA and nucleus combination, on the basis of the LIS produced on at least one NMR signal belonging to said exogenous or endogenous substance;
b) identifying a set of possible candidates for said SA, on the basis of the CC/s in which they distribute; and
c) selecting said SA and nucleus combination, on the basis of the information gathered from steps (a) and (b).

3. The method according to claim 1 wherein step 2) is carried out by:
d) acquiring the MAS-NMR spectrum of the in vitro sample containing the exogenous or endogenous substance under investigation and determining the marker^{EXO} or marker^{ENDO} signal/s;
e) adding a suitable amount of the selected SA to the above in vitro sample, so as to induce a significant LIS of marker^{EXO} or of marker^{ENDO} signal/s, and re-acquiring the same MAS-NMR spectrum; and
f) comparing the marker^{EXO} or the marker^{ENDO} signal/s of steps (d) and (e) and determining in which Cellular Compartment the exogenous or endogenous substance is present.

4. The method according to claim 1, where cellular uptake of exogenous substances is determined.

5. The method according to claim 4 wherein the exogenous substance is any substance not naturally occurring in a biological sample.

6. The method according to claim 5 wherein the exogenous substance comprises exogenous organic substances or exogenous metals or metal ions which NMR signals can be observed.

7. The method according to claim 6 wherein the exogenous substance is selected from the group consisting of: drugs for human and veterinary use, diagnostic and therapeutics agents, contrast agents for imaging techniques, radio-sensitizers for photodynamic and neutron capture therapy, pesticides, herbicides, fertilizers, food additives, preservatives, cosmetics, colorants, waste products, pollutants, and chemicals.

8. The method according to claim 1 wherein the endogenous substance comprises any substance resulting from normal or pathological biochemical processes of cells and tissues.

9. The method according to claim 8 wherein the endogenous substance is selected from the group consisting of natural carbohydrates, urea, lactate, citrate, acetate, carbonate, malonate, choline, creatine, phosphate, piruvate and natural amino acids.

10. The method according to any one of claims 1 to 3, wherein the SA is selected from compounds containing a metal ion of the lanthanide group including: Ce³⁺; Pr³⁺; Nd³⁺; Pm³⁺; Sm³⁺; Eu³⁺; Tb³⁺; Dy³⁺; Ho³⁺; Er³⁺; Tm³⁺; and Yb³⁺.

11. The method according to claim 10 wherein the SA comprises lanthanide complexes of ligands selected from: EDTA (ethylenediaminetetraacetic acid); PCTA (3,6,9,15-tetraazabicyclo-[9.3.1]-pentadeca-1(15)11,13-triene-3,6,9-tris(methane phosphonic)acid); BOPTA ((4RS)-[4-carboxy-5,8,11-tris(carboxymethyl)-1-phenyl-2-oxa-5,8,11-triazatridecan-13-oic acid]) or derivatives thereof; DTPA (diethylenetriamine pentaacetic acid) or derivatives thereof; DOTA (1,4,7,10-tetraazocyclo-dodecane-N,N′,N″,N″″-tetraacetic acid) or derivatives thereof; DO3A (1,4,7,10-tetra azacyclododecane-1,4,7-triacetic acid) or derivatives thereof; DOTP (1,4,7,10-tetraazacyclododecane-1,4,7,10-tetrakis(methane phosphonic)acid or derivatives thereof; and ([3β(R),5β, 12α]-3-[[4-[bis[2-bis(carboxymethyl)amino]-ethyl]amino]-4-carboxy-1-oxobutyl]amino]-12-hydroxycholan-24-oic acid).

12. The method according to any one of claims 1 to 3, wherein the cell sample is selected from human or animal cells, cells cultures, tissues and organ cells, vegetal cells, part of trunks, leaves and food cells of both animal or vegetal origin.

13. The method of claim 1 for use in the fields of medicine, diagnostics, photodynamic and neutron capture therapy, pharmacology and pharmacokinetics, toxicology, cosmetics, food preservation, and botanics.

14. The method according to any one of claims 1 to 3, wherein the kinetic parameters of cellular uptake are determined.
